(19) United States
(12) Patent Application Publication
(10) Pub. No.: US 2024/0369524 A1
(43) Pub. Date: Nov. 7, 2024

(54) Automatic Classification of Excavation Materials

(71) Applicant: Queen's University at Kingston, Kingston (CA)

(72) Inventors: Unal Artan, Kingston (CA); Heshan Fernando, Kingston (CA); Joshua Marshall, Kingston (CA)

(21) Appl. No.: 18/285,722

(22) PCT Filed: Apr. 6, 2022
(86) PCT No.: PCT/CA2022/050520

§ 371 Date: Oct. 5, 2023

Related U.S. Application Data
(60) Provisional application No. 63/171,688, filed on Apr. 7, 2021.

(51) Int. Cl. G01N 33/24; E02F 9/26

(52) U.S. Cl. CPC G01N 33/24; E02F 9/26

(57) ABSTRACT

Methods and apparatus for automatic material classification use proprioceptive sensing data acquired from equipment interacting with the material. Embodiments enable automatic material identification with low operational complexity and computational overhead. Automatic material classification may be used to improve autonomous operation of robotic excavators, as well as provide useful knowledge about excavation materials (e.g., rock size distribution) in fields such as civil and mining operations, military operations, aggregate material handling and processing, and space exploration and development to improve downstream processing and operations.

### FIELD

The invention relates generally to the field of excavation material classification. Specifically, the invention relates to automatic classification of excavation materials through processing and classification of proprioceptive sensor data that are acquired from the physical interaction between a machine tool and the excavation material. Applications for automatic classification of excavation material include underground and surface mining, construction, aggregate material handling and preparation, and space exploration and development.

### BACKGROUND

Acquiring meaningful knowledge about excavation material characteristics, such as material type, density, cohesion, and size distribution, can optimize upstream and downstream operations in fields such as construction, mining, aggregate material handling and preparation, and space exploration and development. Furthermore, information about the material characteristics could also be used to adapt vehicle controllers for autonomous excavation (see International Patent Application Publication No. WO 2015/109392A). Though some properties, such as rock sizes, can be determined visually using exteroceptive sensors (e.g., cameras and laser scanners), common issues of occlusion and poor lighting conditions (e.g., underground) and dust can limit the performance of vision-based methods (e.g., International Patent Application Publication No. WO 2017/100903A, U.K. Patent No. GB 2536271B).

Previous work has proposed automation of certain aspects of excavation. For example, Khorzoughi and Hall (International Journal of Mining, Reclamation and Environment 29 (5): 380-390, 2015) used vibration measurements to characterize a diggability metric to support excavation equipment selection, while Zauner et al. (Automation in Construction 109:1-9, 2020) used cylinder position measurements, wheel velocity measurements, and throttle input signals to classify wheel loader work cycles. However, automatic excavation material classification was not investigated.

### SUMMARY

One aspect of the invention relates to a method for classifying excavation media, comprising: obtaining sensor signals from one or more proprioceptive sensors on a machine interacting with the material; using a processor to process the sensor signals, wherein processing includes extracting features from the sensor signals; selecting one or more classification categories corresponding to physical characteristics of the material; using the extracted features as inputs to a classifier; wherein the classifier uses one or more algorithms to classify the identified features into the selected classification categories; and outputting a result indicating at least one classification category relating to a physical characteristic of the material.

In embodiments, the excavation media comprises fragmented rock, gravel, sand, soil, or mixtures thereof.

In embodiments, the one or more proprioceptive sensor comprises at least one of a force sensor, a pressure sensor, an inertial measurement unit (IMU) sensor, a displacement (linear, angular) sensor, a current sensor and a voltage sensor.

In embodiments, the machine comprises an excavator, haulage equipment, a load haul dump (LHD) machine, or a conveyor.

In embodiments, the machine is operating in an application selected from underground mining, surface mining, construction, material handling, material preparation, and space exploration and development.

In embodiments, the machine interacts with the material manually, partially autonomously, or fully autonomously.

In embodiments, processing the sensor signals and extracting features includes an analysis with respect to time, frequency, amplitude, or a combination thereof.

In embodiments, processing the sensor signals and extracting features includes a statistical analysis, a stochastic analysis, a fractal analysis, a wavelet analysis, a spectral analysis, or a combination of two or more thereof.

In one embodiment, the classifier uses supervised learning to classify the identified features according to the selected classification categories.

In one embodiment, the classifier uses unsupervised learning to classify the identified features according to the selected classification categories.

In one embodiment, the classifier uses unsupervised and supervised learning to classify the identified features according to the selected classification categories.

Embodiments may further comprise obtaining and processing sensor signals from one or more exteroceptive sensors. In embodiments, the one or more exteroceptive sensors may be selected from cameras and laser scanners and combinations thereof.

Another aspect of the invention relates to an apparatus for classifying material, comprising: an input device that receives at least one sensor signal from at least one proprioceptive sensor of a machine interacting with the material; a processor that: processes the at least one sensor signal and extracts features in the at least one sensor signal; selects one or more classification categories corresponding to physical characteristics of the material, identifies extracted features of the at least one sensor signal that are similar for each selected classification category of the material; uses the identified features as inputs to a classifier that uses an algorithm to classify the identified features into the selected classification categories; and outputs a result indicating at least one classification category relating to a physical characteristic of the material.

In various embodiments, the excavation media comprises fragmented rock, gravel, sand, soil, or mixtures thereof.

In various embodiments, the at least one proprioceptive sensor comprises a force sensor, a pressure sensor, an inertial measurement unit (IMU), a displacement (linear, angular) sensor, a current sensor, or a voltage sensor.

In various embodiments, the machine comprises an excavator, wheel loader, haulage equipment, a load haul dump (LHD) machine, or a conveyor.

In various embodiments, the processor processes the sensor signals and extracts features based on an analysis with respect to time, frequency, amplitude, or a combination thereof.

In various embodiments, the processor processes the sensor signals and extracts features using a statistical analysis, a stochastic analysis, a fractal analysis, a wavelet analysis, a spectral analysis, or a combination of two or more thereof.

In one embodiment, the classifier uses supervised learning to classify the identified features according to the selected classification categories.

In one embodiment, the classifier uses unsupervised learning to classify the identified features according to the selected classification categories.

In one embodiment, the classifier uses unsupervised and supervised learning to classify the identified features according to the selected classification categories.

In various embodiments, the apparatus further comprises one or more exteroceptive sensors. In various embodiments, the one or more exteroceptive sensors may be selected from cameras and laser scanners.

Another aspect of the invention relates to a non-transitory computer readable storage media compatible with a computer, the storage media containing instructions that, when read by the computer, direct the computer to carry out processing steps comprising one or more of: processing sensor signals from one or more proprioceptive sensors disposed on a machine interacting with a material; wherein processing includes extracting features from the sensor signals; selecting one or more classification categories corresponding to physical characteristics of the material; using the extracted features as inputs to a classifier; wherein the classifier uses one or more algorithms to classify the extracted features into the selected classification categories; and outputting a result indicating at least one classification category relating to a physical characteristic of the material.

In one embodiment the non-transitory computer readable storage media may include a classifier that uses one or more algorithms selected from a supervised learning algorithm and an unsupervised learning algorithm, or a combination thereof.

In one embodiment the non-transitory computer readable storage media may include a classifier that uses one or more supervised learning algorithm selected from a k-nearest neighbour (KNN) algorithm and an artificial neural network (ANN) algorithm, or a combination thereof.

### BRIEF DESCRIPTION OF THE DRAWINGS

For a greater understanding of the invention, and to show more clearly how it may be carried into effect, embodiments will be described, by way of example, with reference to the accompanying drawings, wherein:

FIG. 1 is a block diagram showing a generalized process for automatic material classification, according to one embodiment.

FIG. 2 is a schematic diagram showing placement of proprioceptive force and inertial measurement sensors on the loading mechanism of a wheel loader or a load haul dump machine, according to embodiments described in Examples 1 and 2.

FIG. 3 is a schematic diagram showing placement of proprioceptive inertial measurement sensors on a haulage equipment, according to one embodiment, which is described in Example 3.

FIGS. 4A (upper panel) and 4B (lower panel) are plots of force signals obtained from excavation trials with an Epiroc ST14 load haul dump machine digging and loading rock (upper panel, 42 trials) and gravel (lower panel, 44 trials) materials.

FIGS. 5A-5C are plots of a subset of the force sensor signals of FIGS. 4A and 4B after processing.

FIG. 6 is a schematic diagram of a model developed to test supervised and unsupervised machine learning for classification of materials, according to one embodiment, which is described in Example 1.

FIG. 7 is a bar graph showing classification accuracies of six classification algorithm structures using five different feature sets derived from the data of FIGS. 5A-5C, wherein each bar indicates average classification accuracy of 50 independent runs for each algorithm, and the error bars indicate standard deviations.

FIG. 8 is a schematic diagram of an artificial neural network (ANN) model developed for binary classification of material, according to an embodiment described in Example 1.

FIGS. 9A and 9B are decision boundary plots for k-nearest neighbour (k-NN) classification of rock and gravel in Example 1, for 1-NN and 5-NN decision boundaries, respectively.

FIG. 10 is a plot showing clustering results of a binary classifier based on a k-means unsupervised machine learning in Example 1.

FIGS. 11A-11D are plots showing an example acceleration signal and control input signals recorded during manual excavation trials in Example 2.

FIGS. 12A and 12B are plots showing bucket cylinder extension and the absolute value of the time derivative of the acceleration to estimate the start and stop times of the excavation cycle during manual excavation in Example 2.

FIGS. 13A-13C are plots of wavelet feature sets extracted from acceleration signals a_{1,x}, from a_{2,x}, and a_{3,x}, respectively, obtained from sensors on a wheel loader as described in Example 2.

FIGS. 14A-14C are plots of the estimated size distributions of Granular B and Rock using wavelet features extracted from acceleration signals a_{1,x}, from a_{2,x}, and a_{3,x}, respectively, obtained from sensors on a wheel loader as described in Example 2.

FIG. 15 is a plot of fragmentation measurements and estimated size distribution models for rock Piles 1-4 of Example 3.

FIGS. 16A and 16B are plots showing acceleration and the absolute value of the time derivative of the acceleration to estimate the start and stop times of the loading cycle in Example 3.

FIGS. 17A and 17B are plots of wavelet feature sets extracted from acceleration signals a_{1,z}, and a_{2,z}, respectively, obtained from sensors on a haulage equipment as described in Example 3.

FIGS. 18A and 18B are plots of the estimated size distribution of Piles 2-4 using wavelet features extracted from acceleration signals a_{1,z}, and a_{2,z}, respectively, obtained from sensors on a haulage equipment as described in Example 3.

FIG. 19 is a plot of wavelet feature sets extracted from force signals, obtained from sensors mounted on a 1-tonne capacity wheel loader and a 14-tonne capacity load haul dump machine as described in Example 4.

FIG. 20 is a plot of the estimated size distribution of Granular B using wavelet features extracted from force signals, obtained from sensors mounted on a 1-tonne capacity wheel loader and a 14-tonne capacity load haul dump machine as described in Example 4.

FIG. 21 is a plot of the estimated size distributions for Rock, Muck and Gravel using wavelet features extracted from force signals, obtained from sensors mounted on a 1-tonne capacity wheel loader and a 14-tonne capacity load haul dump machine as described in Example 4.

### DETAILED DESCRIPTION OF EMBODIMENTS

Described herein are methods and apparatus that provide automatic classification of excavation materials using proprioceptive sensor data that are collected from interaction between a machine and the material. Proprioceptive sensors measure values internal to excavation and haulage equipment such as vibrations (e.g., measured using accelerometers), rotations (e.g., measured using gyroscopes), pressures (e.g., measured with pressure transducers), and cylinder extensions (e.g., measured with wire potentiometers). As described herein, using propriocepitve sensors overcomes the drawbacks of exteroceptive sensors as the sensors capture signals generated during the equipment-material (i.e., tool-material) interaction.

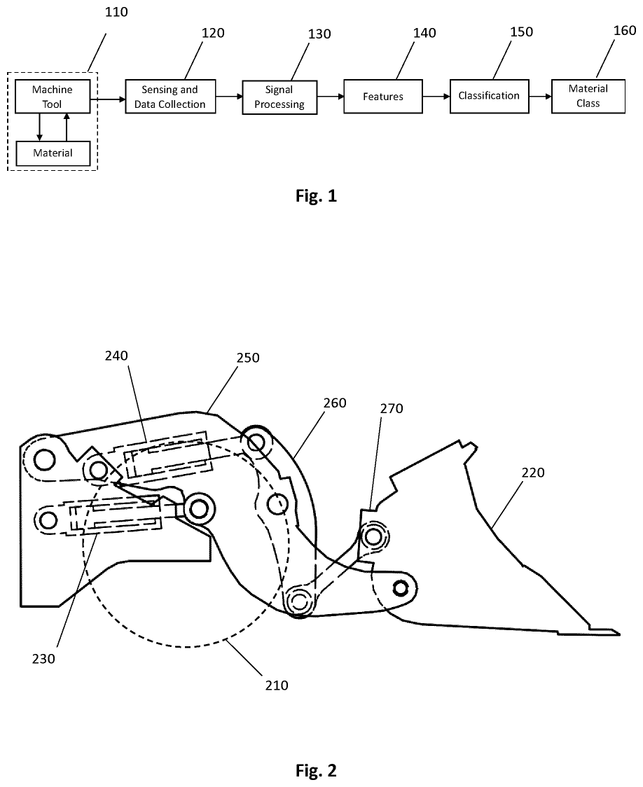

A generalized process according to one embodiment is shown in FIG. 1, which includes machine tool-material interaction 110, sensing and data collection 120, signal processing 130, feature extraction/selection 140, classification 150, to produce an output 160 indicative of the material class.

Embodiments may include a data processing system in which one or more of data collection 120, signal processing 130, feature extraction/selection 140, classification 150, and producing an output are implemented. The data processing system may include one or more computers and be implemented onboard the machine, or the data processing system may be distributed such that parts of it are onboard the machine and other parts are located remotely, e.g., in a base station. The data processing system may include a user interface (e.g., a graphical user interface (GUI)) to allow receiving user input and extraction of information such as system status, data, etc. The data processing system may be, for example, a server system or a personal computer (PC) or tablet-based system. The data processing system may include one or more of an input device such as a mouse, touchpad, and/or keyboard, a processor (e.g., one or more central processing unit (CPU)), memory, a display device, and an interface device including one or more network connections. The data processing system may be adapted for communicating with the onboard sensors and optionally other data processing systems over a network via the interface device. For example, the interface device may include an interface to a network such as the Internet and/or another wired or wireless network (e.g., a wireless local area network (WLAN), a cellular telephone network, via a suitable wireless communications network and protocol, for example, those based on WiFi, Bluetooth, GSM, CDMA, UMTS, LTE, etc.).

The CPU may include or be operatively coupled to dedicated coprocessors, memory devices, or other hardware modules. The CPU is operatively coupled to the memory including non-transitory computer readable media which stores an operating system for general management of the system and instructions (i.e., software) that direct the computer to carry out processing steps. The CPU is operatively coupled to the input device for receiving user commands or queries and for displaying the results of these commands or queries to the user on the display. Commands and queries may also be received via the interface device and results may be transmitted via the interface device. The data processing system may include a database system (or storage) for storing data and programming information. The database system may include a database management system and a database and may be stored in the memory of the data processing system. In general, the data processing system includes non-transitory computer readable media with stored programmed instructions which when executed by the processor cause certain steps of the embodiments described herein, e.g., certain steps of FIG. 1, to be performed. For example, the instructions may be associated with one or more of data collection 120, signal processing 130, feature extraction/selection 140, classification 150, and producing an output 160 of FIG. 1. Of course, the data processing system may contain additional software and hardware, a description of which is not necessary for understanding the invention.

Embodiments may include non-transitory computer readable storage media for use with a computer, processor, etc., the storage media having instructions stored thereon that, when read by the computer, direct the computer to carry out processing steps corresponding to one or more of data collection 120, signal processing 130, feature extraction/selection 140, classification 150, and producing an output 160, as shown in FIG. 1.

Embodiments may enhance a control system of an autonomous or robotic excavator by providing material classification during digging and loading. For example, material class information may be used to adapt control parameters of an autonomous excavation system online or after each excavation pass (e.g., using a lookup table or local optimization). Such adaptation may improve bucket filling consistency in autonomous excavation, for example. The ability to classify excavation material autonomously may be useful in fields such as civil and mining engineering and aggregate material handling and preparation, to optimize operations such as blast designs, downstream rock crushing and/or conveying systems, and in remote operations, for example hazardous material handling and space exploration and development.

It will be appreciated that embodiments described herein may be implemented on equipment with varying degrees of automation, from fully autonomous (i.e., robotic excavators, autonomous haulage trucks) to partially autonomous (e.g., only the dig or bucket-filling operation is automated), to fully manual. Classifying excavation media may improve efficiency of downstream operations, e.g., by providing an operator with an indication of the classification of the excavated material and consequently removing ambiguity about next processing steps. Further, although embodiments are described primarily with respect to surface and underground mining, it will be understood that they are broadly applicable to other fields such as, but not limited to, construction, civil applications, military applications, aggregate handling and preparation, and space exploration/development.

Embodiments described herein overcome difficulties in explicitly modelling heterogenous excavation materials by using data-driven methods, such as but not limited to, machine learning for identifying different classes, which can be used for example in autonomous excavation. Systems using exteroceptive sensing (i.e., sensing the environment remotely) based on vision (e.g., using cameras) and laser scanners (e.g., LiDAR) for estimating size distribution of blasted rock are known (e.g., WO 2017/100903A, GB 2536271B). However, vision-based systems only see the surface of the material and are usually not feasible in underground scenarios. Cameras, in particular, suffer from challenges with poor lighting underground. In contrast, proprioceptive sensing (i.e., sensing the physical interaction between the tool and the environment), as employed in embodiments described herein, overcomes the limitations of exteroceptive sensing by “seeing” through, or below the surface of the material, resulting in a powerful approach for excavation material characterization and/or identification during the tool-material interaction.

It will of course be appreciated that the invention is not limited to only the type and placement of proprioceptive sensors described herein. Other embodiments may combine data from proprioceptive sensing with data from exteroceptive sensing such as cameras and laser scanners.

Various features of embodiments are described below, by way of non-limiting examples, with reference to the generalized embodiment of FIG. 1.

### Machine Tool-Material Interaction

According to embodiments, data may be collected from various types of machine tool-material interactions, including different types of machines and different types of materials. A machine may include, but is not limited to, equipment such as a front-end wheel loader, hydraulic or electric excavator, wheeled backhoe, haulage truck, rail car, and both stationary and mobile crushing and/or conveying systems. A tool may include any part of the machine that physically interacts with the material, such as, but not limited to, a bucket, shovel, bed, conveyor belt, etc.

Material may include, but is not limited to, soil, sand, gravel, fragmented rock, aggregate, or any combination there of.

One embodiment includes the bucket (tool) of a front-end wheel loader as used in construction and surface mining, or a load haul dump machine as used in underground mining, interacting with a pile of material while digging and loading material. An implementation is shown in the embodiment of FIG. 2, which depicts the front portion of a load haul dump machine. Referring to FIG. 2, components shown include the front wheel(s) 210 and bucket 220 with actuating mechanism including hydraulic lift cylinder 230, hydraulic dump cylinder 240, boom 250, and Z-bar linkage 260. According to embodiments, one or more sensors may be disposed on various components to capture force, position, and/or vibration data during digging and loading material. For example, a pressure sensor may be disposed on the lift cylinder 230 to obtain force measurements, a position sensor may be disposed on the dump cylinder 240 to obtain cylinder extension measurements, and one or more inertial measurement unit (IMU) may be placed on the bucket 220 (e.g., at 270) and/or boom 250 to capture vibration data, during digging and loading material. In these embodiments, the machine may be controlled manually, by tele-remote, or autonomously.

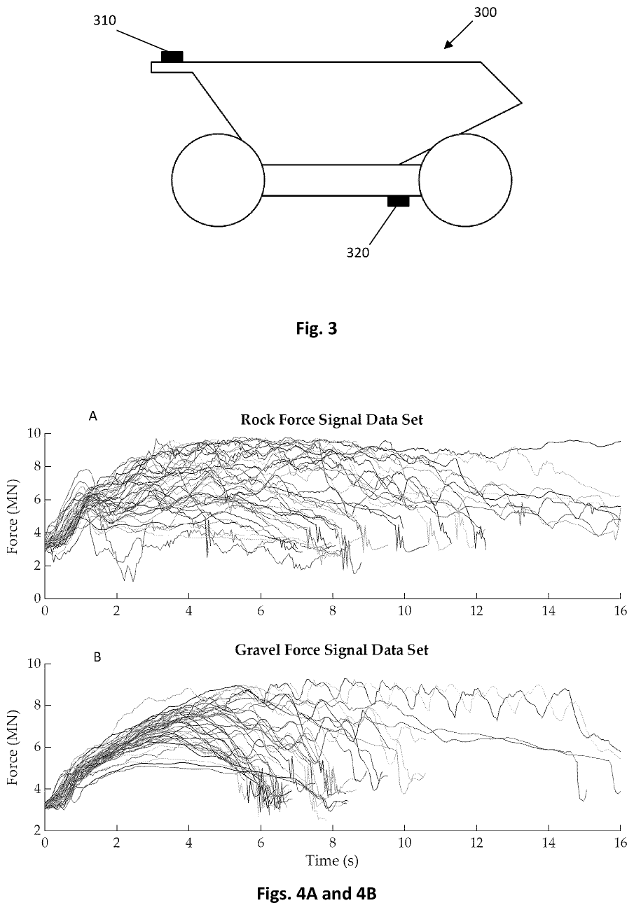

Another embodiment relates to dumping of material into the bed (i.e., tool) of haulage equipment, such as a dump truck, or e.g., an Epirock Minetruck MT-54 as used in underground mining operations, or e.g., a Komatsu 930E-5 haul truck as used in surface operations. Such haulage equipment may be equipped with one or more sensors (e.g., strain sensors, IMUs), physically coupled to the bed so as to produce sensor signals as material is dumped onto the bed, for example, by a hydraulic excavator or by a front end loader. The haulage equipment may be controlled manualy, by tele-remote, or it could be fully autonomous. The equipment loading the material onto the haulage equipment's bed may be operated manualy, by tele-remote control, or it could be fully autonomous. An implementation according to one embodiment is shown in the diagram of FIG. 3, wherein an inertial sensor 310 disposed on the bed and another interial sensor 320 disposed on the chassis obtain data from loading material into the bed of haulage equipment 300. The inertial sensors may be, for example, IMUs.

Another embodiment relates to dumping of material onto a convyer belt, elevator, or other such equipment for transporting material (i.e., the tool). Appropriately placed sensors may produce sensor signals from the conveyor belt or other such tool as it is being loaded with material, and/or as the material is transported.

### Sensing and Data Collection

According to embodiments, proprioceptive sensing is implemented onboard the machine, by disposing one or more hardware proprioceptive sensors on the machine. The one or more sensors produce sensor signals which capture data about the machine tool-material interaction. The data may be stored and processed onboard the machine, or it may be sent to storing and/or processing hardware located off the machine, according to a data processing system, via a suitable wireless communications network and protocol, for example, those based on WiFi, Bluetooth, GSM, CDMA, UMTS, LTE, etc. Proprioceptive sensing may include using one or more sensors to sense one or more of, for example, force, inertia, motion, sound, vibration, and current/voltage. Placement of one or more proprioceptive sensors may be anywhere on the machine, including on the machine tool which interacts with the material. For example, in one embodiment, pressure sensors may be mounted on a load haul dump machine's hydraulic lift cylinders and in another embodiment pressure sensors may be mounted on a wheel loader's lift cylinders. The pressure sensors may be used to measure machine tool-material interaction forces during digging and loading. Such an embodiment is described in detail in Example 1 for a load haul dump machine and described in Example 4 for a wheel loader. In another embodiment, inertial measurement units mounted on the boom and bucket of a wheel loader may be used to measure acclerations during digging and loading material. Such an embodiment is described in detail in Example 2. In another embodiment, sensors such as accelerometers or IMUs may be mounted on haulage equipment, for example on the bed and/or on the chasis, and used to measure vibrations as material is dumped onto the bed of the haulage equipment. Such an embodiment is described in detail in Example 3.

### Signal Processing

According to embodiments, sensor signals (i.e., data) obtained from proprioceptive sensing may be subjected to processing according to a data processing system, to remove bias, segment useful portions, convert the signal into other domains (e.g., time, frequency, spatio-temporal).

According to embodiments, signal processing may include, for example, an analysis with respect to time, frequency, amplitude, or a combination thereof, a statistical analysis, a stochastic analysis, a fractal analysis, a wavelet analysis, a spectral analysis, or a combination of two or more thereof.

The following are non-limiting examples of signal processing that may be implemented in various embodiments.

Segmentation: Sensor signal(s) may be proccessed by segmenting the signal to remove data that is outside the machine tool-material interaction time window. The sensor signal(s) may also be segmented into distinct phases of the machine tool-material interaction.

Time Derivative: Sensor signal(s) may be processed by applying a gradient filter, which computes a time derivative of the signal. In one embodiment, the time derivative of the force signals may be computed using a central-difference numerical gradient method, which computes the derivative of a signal using central difference for interior data points and single sided differences for the edges of the signal.

Frequency Spectrum: Sensor signal(s) may be processed by converting time-domain signals into the frequency domain (ϑ). In one embodiment, the frequency power spectrum |P(ϑ)| of a segmented portion of force signals as described above can be computed using the Discrete Fourier Transform. In one embodiment, a high-pass filter is applied to the force signals to pass signals with a frequency higher than the cutoff frequency, f_{cut}.

### Features

According to embodiments, features may be extracted from raw or processed sensor signals to reduce the data for classification. The goal is to find features that are similar (i.e., invariant) for excavation materials in the same category and different (i.e., distinguishable) for materials in different categories. Highly distinguishgable features may allow the use of simple classifiers (e.g., thresholding) for classification. Alternatively, raw sensor data may be directly inputted to a sophisticated classsifier (e.g., a Deep Learning algorithm) for classification, which would not require a separate feature extractor. The following non-limiting examples describe some embodiments of feature extraction.

Wavelet: Wavelet analysis may be used to process time-domain sensor signals. Wavelet analysis starts with a mother wavelet Ψ(t), which has two distinct properties: 1) the wavelet has zero mean and 2) has a Euclidean norm of one. The mother wavelet can be scaled by s and translated by t to produce

$${Ψ}_{s , τ} ( t ) = \frac{1}{\sqrt{s}} Ψ ( \frac{t - τ}{s} ) . \qquad ( 1 )$$

There are an infinite number of wavelets that can be constructed and selecting wavelets that have similar shape (waveform) to a signal may be desired. According to embodiments, a specific waveform is not required, rather the same waveform is used throughout the signal processing.

The wavelet transform, specifically the continuous wavelet transform (CWT), captures both spectral and temporal information hidden within the signal. The CWT uses a wavelet, Ψ_{s,τ}(t), and computes the convolution of this wavelet with the signal. The CWT for an arbitrary wavelet is given as

$$γ ( s , τ ) = \int f ( t ) {Ψ}_{s , τ}^{*} ( t ) dt , \qquad ( 2 )$$

where Ψ^{*} denotes the complex conjugate of Ψ, and f(t) the input signal.

An approximate frequency (pseudo-frequency) is estimated using the relationship

$${f}_{s} = \frac{{f}_{c}}{s \cdot T} , \qquad ( 3 )$$

Where s is the scale, fₛ is the frequency in Hz for a given scale s, f_{c} is the center frequency of the wavelet and T is the sampling period. Using this relationship γ(s, τ) is mapped to γ(f, τ). For example, in one embodiment the input signals are accelerations and in another embodiment the input signals are forces. The output of the wavelet analysis γ(f, τ) may be processed further to extract a frequency result, i.e., a spectrogram by using the mean wavelet value at each frequency f by,

$$β ( f ) = \frac{1}{Δτ} {\int }_{{τ}_{1}}^{{τ}_{2}} γ ( f , τ ) d τ , \qquad ( 4 )$$

where τ₁ and τ₂ are the start and end time of the tool material interaction respectively and Δτ is the difference between τ₂ and τ₁. Equation (4) may be processed further to standardize or normalize the features using, for example, the total mass of the material, M, such that,

$$\tilde{β} ( f ) = \frac{β ( f )}{M} . \qquad ( 5 )$$

Mean and Standard Deviation: Means and standard deviations may be extracted as features x from force signals f, optionally with pre-processing (e.g., one or more of time derivative. Power spectrum, and wavelet analyses). In one embodiment, the mean of a signal (g[p, q]) may be computed as

$$mean ( g [ p , q ] ) = μ = \frac{1}{q - p} {\sum }_{i = p}^{q} ( g [ i ] ) \qquad ( 6 )$$

and the standard deviation of that signal may be computed as

$$std ( g [ p , q ] ) = \sqrt{\frac{1}{q - p} {\sum }_{i = p}^{q} {( g [ i ] - μ )}^{2}} . \qquad ( 7 )$$

Peak Frequency: In some embodiments, time-domain sensor signals may be processed and converted to frequency domain, such as in the case of wavelet analysis, and the peak frequency ϑ₁ can be extracted as a feature. For example, in one embodiment using force signals, the frequency with the highest peak in the force signal's power spectrum P(ϑ) may be extracted as a feature. The highest peak may be found by first finding all of the local peaks of the frequency spectrum and computing the largest value. A local peak may be defined as a data sample that is larger than its two neighbouring samples.

### Classification

As used herein, the term “classification” may include one or more of identification, classification, and characterization. According to embodiments, classification may be performed by a classifier algorithm that uses as inputs raw sensor signals, processed sensors signals, features, or any combination thereof, and outputs a material class. The sophistication of the classification algorithm required depends on the sophistication of the signal processing and feature extraction. The classifer may vary from basic thresholding to advanced supervised and unsupervised learning algorithms, which may include Deep Learning algorithms. Supervised learning minimizes an error/cost based on differences between targets and outputs (correct responses). Unsupervised learning is about clustering data when prior training data is not available. For material classification, in which all possible classes of materials may not be known at the time of training, unsupervised learning methods may perform well. As described herein, either or both types of machine learning may be implemented in embodiments.

Supervised Learning using K-Nearest Neighbours (KNN): The k-nearest neighbour (KNN) is an example of a supervised learning algorithm that may be used in according to embodiments described herein. The KNN algorithm categorizes a new input x′ by assigning it the label of the majority samples x among its k nearest samples (with k odd to avoid ties). The algorithm is initialized using a set of training data whose class labels are known. The nearest neighbours of a new input x′ are determined using a distance metric. For example, the Euclidean distance ||x′−xᵢ||² may be used.

Example 1 describes an implementation of a classifier based on a KNN algorithm with force sensor data to classify excavation materials.

Supervised Learning using Artificial Neural Networks: A feedforward multi-layer perceptron (MLP) network, trained using supervised gradient descent methods such as backpropagation, is an example of artificial neural network (ANN) based supervised learning that may be used in accordance with embodiments described herein for pattern recognition and classification. One configuration is a three-layer network, which has an input layer, an output layer, and a hidden layer. The units in each layer are interconnected by modifiable weights w in a feedforward configuration. Thus, signals are processed in one direction, from input to output. Furthermore, there is a single bias unit b, which serves as a threshold for activating each unit, connected to every unit other than the input units.

The number of input units is determined by the number of input features m, and the number of output units is determined by the number of classes c. The number of hidden units n_{H} is a design parameter. The input layer passes through an m-dimensional input feature vector to each unit in the hidden layer.

Example 1 describes an implementation of a classifier based on a MLP network with force sensor data to classify excavation materials.

Unsupervised Learning with k-Means: The unsupervised learning algorithm k-means is an example of an unsupervised learning algorithm that can be used according to embodiments described herein. The k-means algorithm partitions n observations x₁, x₂, . . . ,Xₙ into k clusters. To begin, k centroids (or means) (μ₁, . . . ,μₖ are initialized in the input space. For example, this can be done by randomly choosing k observations from the data set. The value of k may be assigned based on the final application (e.g., the number of desired classes).

In one embodiment the clustering algorithm may be implemented as follows. First, each observation x is classified to the nearest centroid μⱼ using a distance metric such as Euclidean distance ||xᵢ−μⱼ||². After all n observations are classified, the centroids are recomputed as the means of their clusters

$${μ}_{j} = \frac{1}{{N}_{j}} {\sum }_{i = 1}^{{N}_{j}} {x}_{i}$$

where Nⱼ is the number of observations belonging to the cluster j. The algorithm is iterated until no observation reassignments are made and the centroids stop moving.

Example 1 describes an implementation of a k-means classifier with force sensor data to classify excavation materials. Example 2 and Example 3 describe implementations of a k-means classifier with inertial measurement data to classify excavation materials.

### Material Classes

According to embodiments, excavation materials may be classified into two or more of numerous categories (e.g., based on type, mechanics, rock size, fragmentation, etc.). The output material classes may be dictated by the needs of an application. For example, output material classification based on material type could be used to update the parameters of an autonomous digging controller (e.g., using a lookup table or local optimization) based on material type (e.g., gravel or rock). Alternatively, output material classification based on particle size distribution could be used to optimize material processing, such as a crushing and grinding circuit in a mining operation.

Material Type: In some embodiments, sensor signals obtained from machine tool-material interaction may be used for binary classification, for example, classification of rock and gravel materials. Binary classification could be used to adapt the parameters of an autonomous digging controller. This may be implemented by using the binary output and a look up table to select the appropriate parameters based on the classification result.

In one embodiment, acceleration signals obtained from the bed of haulage equipment such as a mining truck (e.g., as described in Example 3 using a scaled model) may be used for multi-class classification of aggregate materials with four different size distributions. The classification could be used, e.g., to direct downstream material processing, or to adapt the parameters of an autonomous digging controller as described above.

Size Distribution: In some embodiments, sensor signals obtained from machine tool-material interaction may be used to classify the material based on size distribution. Embodiments may be based on a size distribution model. A non-limiting example of such a model is the Rosin-Rammler equation, which was used used in an embodiment described herein.

The Rosin-Rammler equation is given by

$$P ( x ) = 1 - {2}^{- {( x / {x}_{50} )}^{n}} , \qquad ( 8 )$$

where P(x) represents the cumulative mass fraction of rocks with size less than x, x₅₀ is the mean passing size and n is the uniformity parameter. Given a data set containing P(x) and x, x₅₀ and n can be found by fitting a line log(−log(1−P(x))/log(2))=m log(x)+b to the data using linear regression. The uniformity parameter is then the slope of the line (i.e., n=m) and x₅₀=e(−b/n).

In order to obtain a ground truth reference, material(s) may be assessed manually and/or with a screening tool and/or with a fragmentation analyis system which utilizes exteroceptive sensors, such as cameras, to generate the model parameters x₅₀ and n.

Machine Response from Tool-Material Interaction: During excavation, the tool imparts a force on the material (e.g., rocks) to change the rock's velocity from an initial velocity to that of the tool. The momentum transfer from the tool to the rocks induces a motion (displacement, velocity, and acceleration) in the tool, which the proprioceptive sensors capture. Research conducted on the transverse impact of spheres (i.e., as a generalized rock shape) on plates (i.e., as part of a tool) yielded the result that the plate motion or response from the collision is dependent on the size of the sphere and the initial velocity. For example, the tool response (displacement), g₂(t) due to a sphere of size d₂ would be similar to the response (displacement) due to a sphere of size d₁, g₁ (t) but scaled in magnitude by d₂/d₁, such that 9₂ (t)≅(d₂/d₁)g₁(t). This assumes the two spheres have the same intial velocity and physical properties. The velocity and acceleration of the tool response, ġ(t) and \ddot{g}(t), respectively also have the relationship as shown above where ġ₂(t)≅(d₂/d₁)ġ₁(t) and \ddot{g}₂(t)≅(d₂/d₁)\ddot{g}₁(t).

This relationship can be leveraged using wavelet features such as those described above because they are created by using signals from proprioceptive sensors that capture the tool response, as discussed above. An estimate of the mean rock size of an unknown rock pile x_{50,U} can then be found using,

$${x}_{50 , u} = ( {\overline{β}}_{U} / {\overline{β}}_{G} ) {x}_{50 , G} , \qquad ( 9 )$$

where β̅_{U} is the mean wavelet feature for an unkown rock pile, β̅_{G} is the ground truth reference mean wavelet feature and x_{50,G} is the ground truth reference mean rock size estimated using one or more of the methods described above.

The uniformity parameter for an unknown rock pile is assumed to be the same as the ground truth reference uniformity parameter n_{G}, such that n_{U}=n_{G}.

Examples 2, 3 and 4 describe implementations of this relationship to estimate the distribution of different rock piles. In each of these examples, the uniformity parameter was assumed to be the same as that of the ground truth uniformity parameter.

All cited publications are incorporated herein by reference in their entirety.

The invention will be further described by way of the following non-limiting Examples.

### Example 1. Force Sensing and Classification with a Load Haul Dump Machine

Use of proprioceptive pressure sensors to measure forces of a load haul dump machine tool-material interaction for material classification is presented here. The material was a rock pile consisting of a mixture of mud, fine gravel, and large fragments of blasted rock (30 to 70 cm in nominal diameter), which is representative of a real rock pile found in underground mining, and a gravel pile consisting mainly of fine dry gravel with smaller rocks and fines. The pressure sensors (Danfoss, measurement range 0 to 350 Bar, analog voltage range 0.5 to 4.5 VDC, accuracy<±0.05% F.S., response time <4 ms, A-D conversion 10-bit) were placed at the head (h) and rod (r) sides of an Epiroc ST14 load haul dump machine's lift cylinders as shown in FIG. 2. These pressure sensors were used to measure the cylinder hydraulic pressures P at the cylinder head side (h) and rode side (r). Using the cylinder head side and rod side cross-sectional areas A, the interaction forces were estimated using f=PₕAₕ−PᵣAᵣ.

FIGS. 4A (upper panel) and 4B (lower panel) show a total of n=86 force signals obtained in this manner (42 from the rock pile and 44 from the gravel pile) with the ST14 machine digging and loading both materials. Each force signal is represented in discrete time as f [k]=f(kT), where k=0,1,2, . . . L is the time-step and T is the sampling time (0.05 s). The signal length L varies because different control parameters may be used (for autonomous digging) or there may be inconsistencies associated with manual digging.

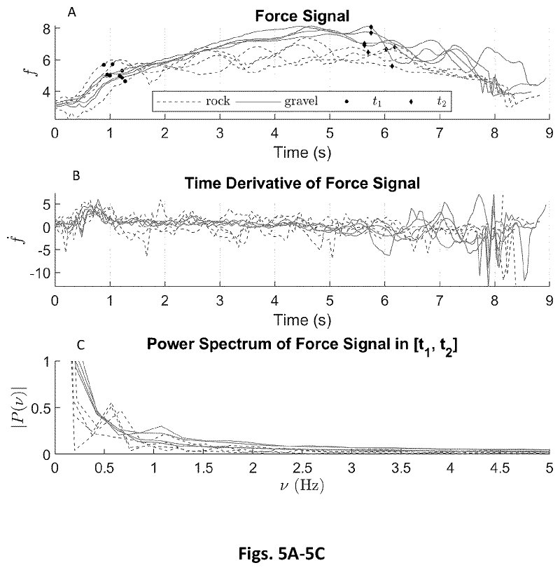

The signals were proccessed by segmenting the signals to remove data that were outside the machine tool-material interaction time window. The sensor signals were then segmented into three phases of the machine tool (i.e., bucket)-material interaction, shown in FIG. 5A (upper panel). The first phase f[0, t₁] is the initial bucket curl, which causes the forces to sharply increase at the start of digging. The second phase f[t₁, t₂] includes most of the digging where the forces evolve due to bucket-material interaction. The third phase f[t₂, L] is the final bucket curl where the forces begin to decrease, which is likely due to material falling to the back of the bucket. Because each of the force signal traces has a different length L, the three phases can be normalized using the dump cylinder extension measurement d. In this embodiment, t₁ is the time-step k where d=0.220 m, and t₂ is the time-step k where d=0.430 m.

The sensor signals were processed by applying a gradient filter, which computes a time derivative of a signal. The time derivative was computed using a central-difference numerical gradient method, which computed the derivative of a signal using central difference for interior data points and single sided differences for the edges of the signal. The computed time derivative of the force signals is shown in the plot of FIG. 5B (center panel).

The sensor signals were also processed by converting time-domain signals into the frequency domain (ϑ) in order to obtain the signal's frequency power spectrum |P(ϑ)|. The frequency power spectrum was computed using the Discrete Fourier Transform. The frequency power spectrum of the force signals for the segmented portion f[t₁, t₂] is shown in the plot of FIG. 5C (lower panel).

Several distinguishing features can be observed in the processed force signals shown in FIGS. 5A-5C. For example, the magnitude and variation of f[t₁, t₂] is much greater in rock compared to gravel. As well, the dominant peak frequency ϑ₁ occurs at a lower frequency for rock compared to gravel. These observations were used to define useful features from the force signals for material classification.

Features such as means and standard deviations were extracted from the processed and segmented force signals using equations (6) and (7). After some inspection and analysis, seven features were selected for classification, as summarized in Table 1.

TABLE 1
Examples of features extracted from force signals

| Feature | Description |
|---|---|
| x₁ | mean(f[0, t₁]) |
| x₂ | std(f[0, t₁]) |
| x₃ | mean(\dot{f}[0, t₁]) |
| x₄ | std(\dot{f}[0, t₁]) |
| x₅ | mean(\dot{f}[t₁, t₂]) |
| x₆ | std(\dot{f}[t₁, t₂]) |
| x₇ | ϑ₁ |

In Table 1, features x₁, x₂, x₃, x₄ were extracted from the first phase f[0, t₁] (bucket curl) of the force signals. These features capture the average magnitude and variation of the forces f and time derivatives of the forces \dot{f} in the initial bucket curl. In this example, these may be distinguishable features as the values of these features may be higher for rock than for gravel. Features x₅ and x₆ capture the average magnitude and variation of \dot{f} in the second phase f[t₁, t₂]. As observed in FIG. 5B, these feature values are expected to be higher for rock than for gravel because there are more fluctuations in the force signals for rock than for gravel.

In Table 1, the feature x₇ is the peak frequency in the force signal power spectrum P(ϑ) (FIG. 5C). No distinguishable features were extracted from the third phase of the force signal f[t₂, L].

Feature Sets: The extracted features were analyzed using visualization tools in MATLAB® (The MathWorks, Inc., Natick, MA, USA) to reduce the feature space. A histogram analysis of the seven features did not reveal any feature that could be used to uniquely discriminate between the rock and gravel materials. The features were further analyzed using a parallel coordinate plot based on the median, the 25 percent quartile, and the 75 percent quartile values for the features. The only three features with no overlap between the two material classes were x₂, x₅ and x₆. Based on this and other selection analyses, five feature sets were formed to evaluate the classification performance.

The first feature set S₁={x₁, x₂, x₃, x₄, x₅, x₆, x₇} included all seven features, second feature set S₂={x₂, x₅, x₆} included the top three features based on the feature analysis, and the third feature set included four features S₃={x₁, x₂, x₃, x₄}, which are the features extracted from the first phase f[0, t₁] of the force signal. The fourth feature set was S₄={x₃, x₆} and the fifth feature set was S₅={x₂, x₅, x₆, x₇}, which are top-ranking features as determined by feature selection algorithms.

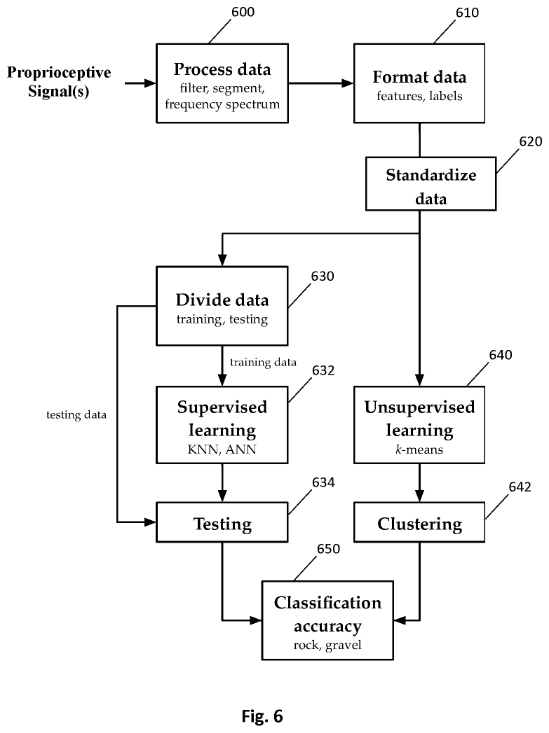

The feature sets were used for training and testing a binary classifier based on a k-Nearest Neighbour (KNN) algorithm, a classifier based on a feedforward multi-layer perceptron (MLP) artificial neural network (i.e., ANN algorithm), and a classifier based on an unsupervised learning algorithm, k-means. A block diagram of an embodiment of the classification method is shown in FIG. 6, at least a portion of which may be implemented in computer software, which shows the intial data processing 600, formatting and feature selection 610, and standardization 620, followed supervised learning 630, 632, 634 or unsupervised learning 640, 642, both of which provide classification, and a final step of evaluation 650. The software may be stored on non-transitory computer readable storage media compatible with a computer, processor, etc., the software including instructions that direct the computer to carry out processing steps corresponding to one or more of intial data processing 600, formatting and feature selection 610, data standardization 620, supervised learning 630, 632, 634 including KNN and/or ANN algorithms such as described below and shown in FIG. 8, or unsupervised learning 640, 642, and evaluation of classification accuracy 650, as shown in FIG. 6.

KNN Classifier: For the KNN classifier, a q-fold cross-validation approach, with q=5, was used to train and test the algorithm. In this approach, the entire input feature set was randomly ordered and split into five groups (i.e., folds). Then each unique group of features was tested after training the KNN algorithm with the remaining four groups. This was repeated for each of the five groups of data. The five testing evaluation results were averaged and reported as the classification performance of the algorithm.

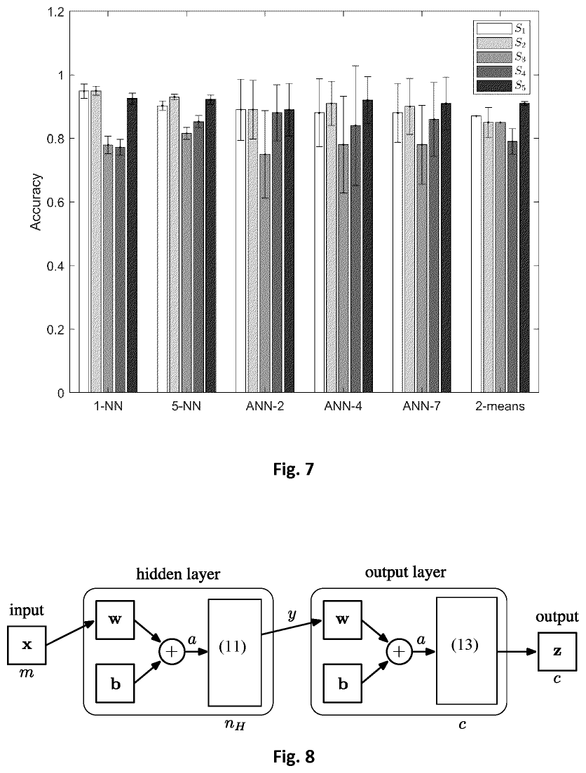

The KNN algorithm was tested with k=1 and k=5 nearest neighbours. The average classification accuracy from 50 independent runs, with different initializations of the data set, is shown in the bar graph in FIG. 7 (bars labelled 1-NN and 5-NN). The standard deviation of the classification accuracies from the 50 independent runs are shown as error bars.

ANN Classifer: For this classifier, a three-layer MLP network was trained and tested for classification of the 7-dimensional feature vectors in Table 1 as gravel or rock materials (i.e., two classes, c=2). The number of input units is determined by the number of input features m, and the number of output units is determined by the number of classes. The number of hidden units n_{H} is a design parameter. The input layer passes through an m-dimensional input feature vector to each unit in the hidden layer. Each hidden unit computes the weighted sum of its inputs to form a scalar net activation

$${a}_{j} = {\sum }_{i = 1}^{m} {x}_{i} {w}_{ji} + b , \qquad ( 10 )$$

where eⱼᵢ denotes the connection weight from input unit i to hidden unit j. Each hidden unit emits an output that is a non-linear function of its activation yⱼ=f(aⱼ). In this example, a sigmoid activation function was used in the hidden units

$${y}_{j} = \frac{1}{1 + {e}^{- {a}_{j}}} . \qquad ( 11 )$$

Similarly, each output unit k computes its net activation based on the hidden signals yⱼ and the connection weights between the hidden units and the output units wₖⱼ, so that

$${a}_{k} = {\sum }_{j = 1}^{{n}_{H}} {y}_{j} {w}_{kj} + b . \qquad ( 12 )$$

In this example a softmax activation function was used for the output layer, calculated as

$${z}_{k} = \frac{{e}^{{a}_{k}}}{{\sum }_{l = 1}^{c} {e}^{{a}_{l}}} , \qquad ( 13 )$$

which transforms the maximum output to 1.0 and reduces all other outputs to 0.0. For rock and gravel material classification, two output nodes were used (i.e., c=2). Each node is trained to output a value of 1.0 for the class, and 0.0 if not.

Once the network configuration was selected, the connection weights were set such that the network provided the desired output for a given input. A backpropagation algorithm was used in this example. The approach is to first randomly initialize the network weights w. Next, the network is presented with input patterns x from a training data set, which are also labelled with the desired target outputs t. For each input pattern, the error between network output z and the target output t is calculated as the mean squared error

$$J ( w ) = \frac{1}{c} {\sum }_{k = 1}^{c} {( {t}_{k} - {z}_{k} )}^{2} \qquad ( 14 )$$

Using a method based on gradient descent, the weights are updated in a direction that will reduce the error

$$Δ {w}_{pq} = - η \frac{\partial J}{\partial {w}_{pq}} , \qquad ( 15 )$$

where η is merely a learning rate that indicates the relative size of the change in weights.

The ANN model used in this example based on equations (10)-(15) is shown schematically in FIG. 8, where (11) and (13) refer to equations (11) and (13) above. The ANN algorithms were tested using a 70/15/15 split for training/validation/testing data. Three networks were tested with hidden layer sizes n_{H}=2, n_{H}=4, n_{H}=7 (labelled ANN-2, ANN-4 and ANN-7, respectively). The average classification accuracy from 50 independent runs, with different initializations of the data set, is shown in the bar graph in FIG. 7 (bars labelled ANN-2, ANN-4, and ANN-7). The standard deviation of the classification accuracies from the 50 independent runs are shown as error bars.

k-means Classifier: For the classifier based on an unsupervised learning algorithm k-means, k=2 was used for the binary classification of rock and gravel materials, using the seven features described in Table 1. The clustering algorithm was implemented as follows. First, each observation x was classified to the nearest centroid μⱼ; using a Euclidean distance ||xᵢ−μⱼ||². After all n observations were classified, the centroids were recomputed as the means of their clusters

$${μ}_{j} = \frac{1}{{N}_{j}} {\sum }_{i = 1}^{{N}_{j}} {x}_{i} \qquad ( 16 )$$

where Nⱼ is the number of observations belonging to the cluster j. The algorithm was iterated until no observation reassignments were made and the centroids stopped moving.

The average classification accuracy from 50 independent runs of the k-means clasifier, with different initializations of the data set, is shown in the bar graph of FIG. 7 (bars labelled 2-means). The standard deviation of the classification accuracies from the 50 independent runs is shown as an error bar. Note that clustering performance in this example, as well as Example 2 and Example 3, is determined using cluster purity Ā∈[0,1], where each cluster ωₖ is asssigned a class cⱼ that is most frequent in that cluster, and the acuracy of this assignment is measured by counting the number of correctly assigned class members in each cluster and dividing by the total number of samples. Thus, more formally

$$\overline{A} ( Ω , C ) = \frac{1}{n} {\sum }_{k = 1}^{K} {max}_{{c}_{j} ∈ C} \vert {ω}_{k} ⋂ {c}_{j} \vert \qquad ( 17 )$$

Performance of the Classifiers and Feature Sets: Overall, high classification accuracies were obtained using the various feature sets and classification algorithms. The highest classification accuracy was 95% using the 1-NN algorithm with both S₁ and S₂ feature sets. The lowest classification accuracy was 75% using the ANN-2 algorithm and the S₃ data set.

The classification accuracies of the five feature sets averaged across the six algorithms were 90%, 90%, 80%, 83%, and 91%, respectively. Given that S₂ and S₅ are subsets of S₁, it is clear that some of the features in S₁ are redundant. The lower performance of S₃ indicates that the features in the first phase of the signal f[0, t₁] are not sufficient to distinguish between rock and gravel. Different features may be required for improved classification early in the digging phase. Another method that would be expected tp perform well for early classification during digging is to extract features from a sliding window of the force signal. S₄ also had lower performance, but this may be due to the feature set's low dimensionality.

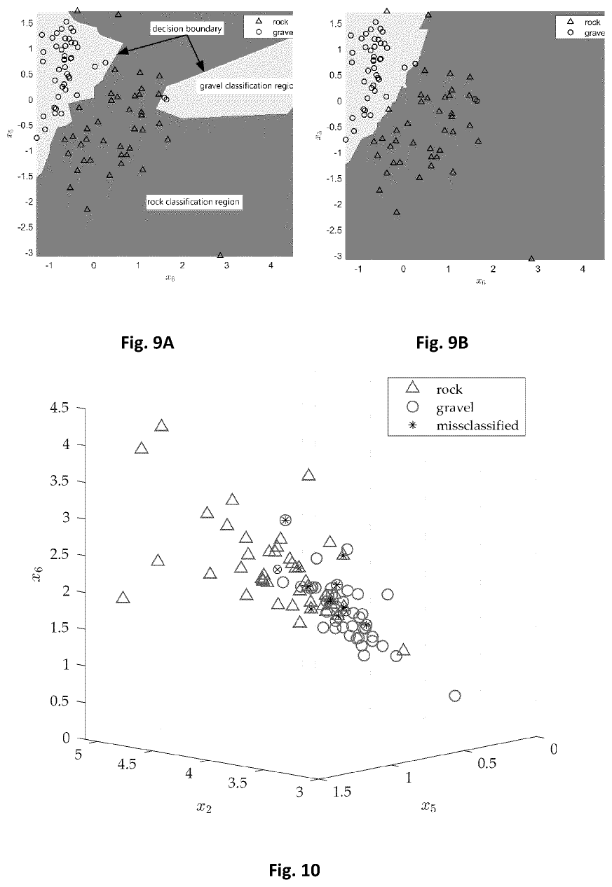

Comparison of Classifiers: The six algorithms were compared using S₂ classification results as a benchmark. Out of the five supervised classifiers, the best performance was achieved with the 1-NN (95% accuracy). However, the 1-NN algorithm may overfit the decision boundaries, as shown in FIG. 9A, which may not be desirable in a classification algorithm. The 5-NN algorithm (93% accuracy) created a more generalized decision boundary, shown in FIG. 9B, which provides more realistic classification results.

The three ANN algorithms had similar classification performance for S₂ (average of 89%); however, their classification performance had more variation in the 50 independent runs compared to other algorithms. This suggests that the ANNs are more sensitive to initialization than other algorithms.

The k-means classifier also achieved good performance (85%) with the S₂ data set. The resulting clusters are shown in FIG. 10. Similar numbers of rock and gravel feature sets are misclassified in this case.

### Example 2. Acceleration Sensing and Wavelet Analysis with a Wheel Loader

This example describes automatic aggregate material classification, using acceleration data obtained from manual excavation with a wheel loader, and wavelet analysis.

### Equipment

Data for material classification were acquired from manual excavation experiments using a wheel loader and three different material piles.

A Kubota R520s wheel loader was instrumented with sensors. The loader has a 1-tonne loading capacity and was equipped with a custom bucket that is similar in design to buckets found on typical mining equipment (e.g., underground load-haul-dump machines). The loading mechanism has a similar configuration to the load haul dump machine configuration shown in FIG. 2. The sensor hardware is described below, and the sensor signals obtained are listed in Table 2.
1) Cylinder Extension: Two wire potentiometers measured the lift cylinder extension θ₁ and dump cylinder extension θ_{d}.
2) Proportional Control Valves: Two electrohydraulic proportional valves control the fluid flow to the lift cylinders and the dump cylinder. The flow rates were proportional to the command signals uₗ=[−1,1] and u_{d}=[−1,1] for the lift cylinders and the dump cylinder, respectively. Here, negative command values corresponded to cylinder retraction and positive command values corresponded to cylinder extension. The command signals were sent by the joystick during manual operation.
3) Throttle: The throttle pedal is actuated by a linear servo motor. The position command to this motor uₜ is a value between 0 and 1, where uₜ=1 corresponds to a fully depressed throttle. Engine RPM was not logged, but the tachometer value could be read by the operator using the dial on the dashboard.
4) Wheel Encoders: Two encoders attached to the front wheels provide the wheel speed measurement v.
5) Inertial Measurement Units (IMU): Three 3-axis Bosch BMI088 IMUs were installed on the boom and bucket to acquire acceleration measurements a=[aₓ, a_{y}, a_{z}] during loading; the gyroscopes were not utilized in this work. The placement of the IMUs are shown in FIG. 2. IMU 1 was installed on the boom, IMU 2 was installed on the left side of the bucket, and IMU 3 was installed on the right side of the bucket. IMU 1 was oriented such that its x-component was along the boom arm. IMU 2 and IMU 3 were oriented such that their x-components were parallel to the base of the bucket. Each IMU was enclosed within an IP67-rated enclosure. The two enclosures on the bucket were mounted beneath thick metal brackets to protect the IMUs from falling rocks.
6) Control System: An onboard control system was used to log sensor data and the actuator command signals. The control system included a main control unit (MCU), seven CAN Peripheral Interface (CPI) sub-control modules, and a laptop running the Robot Operating System (ROS). The MCU operated at 10 Hz for logging sensor data and sending commands via the CPIs. The three IMUs and the joystick were connected through Arduino® microcontrollers to ROS, which logged the acceleration data at 250 Hz.

TABLE 2
Description of signals obtained from
sensors disposed on the wheel loader.

| Signal | Description | Range | |
|---|---|---|---|
| θₗ | lift cylinder extension | [0, 604] | mm |
| θ_{d} | dump cylinder extension | [0, 396] | mm |
| u_{d}, uₗ | lift (l) and dump (d) valve commands | [−1, 1] | |
| uₜ | throttle command | [0, 1] | |
| v | wheel velocity | [0, 3] | m/s |
| a₁, a₂, a₃ | IMU 1, IMU 2 and IMU 3 accelerations | [−24, 24] | g |

### Rock Piles

Three rock piles (Rock, Granular B, Granular A) with different rock size distributions were used for the loading experiments. Each pile contained approximately 35 m³ of material.

Rock: Fragmented limestone with size distribution ranging from fine particles less than 1 mm to large rocks that were 600 mm along the longest length. Analysis produced model parameters of X₅₀=88 mm and n=1.9494. The model parameters were estimated using WipFrag™ (WipWare Inc., North Bay, ON, Canada), a vision-based fragmentation analysis system, to process images of the excavated material after each excavation.

Granular B: Crushed limestone aggregate obtained from a quarry containing particles up to 120 mm in size. Fragmentation analysis using a screening tool produced model parameters of x₅₀=6.1 mm and n=0.6752. Fragmentation analysis using WipFrag® produced model parameters of x₅₀=50.9 mm and n=2.092. A combination of the two size distribution models yields the model parameters x₅₀=19.8 mm and n=0.9333. Combining the two fragmentation analysis methods overcomes the drawbacks of the screening method over estimating the fine region and the image analysis system over estimating the coarse region.

Granular A: Crushed limestone aggregate obtained from a quarry containing the smallest particle sizes out of the three materials with a maximum size of approximately 25 mm. Fragmentation analysis using a screening tool produced model parameters of x₅₀=4.8 mm and n=0.7642. Fragmentation analysis using WipFrag® produced model parameters of x₅₀=23.8 mm and n=3.1213. A combination of the two size distribution models yields the model parameters x₅₀=10.6 mm and n=1.0692. Combining the two fragmentation analysis methods overcomes the drawbacks of the screening method over estimating the fine region and the image analysis system over estimating the coarse region.

### Analyses

A summary of the signals used is provided in Table 2. Equations (1)-(5) were used for the wavelet analysis and the continuous wavelet transform function in MATLAB®. For the analysis a k-means unsupervised learning algorithm (16) was used for the different material classes. Size distribution estimates were generated using equation (9).

A total of 10 trials with the Rock pile, 30 trials with the Granular B pile, and 30 trials with the Granular A pile were analyzed.

### Acceleration Signals

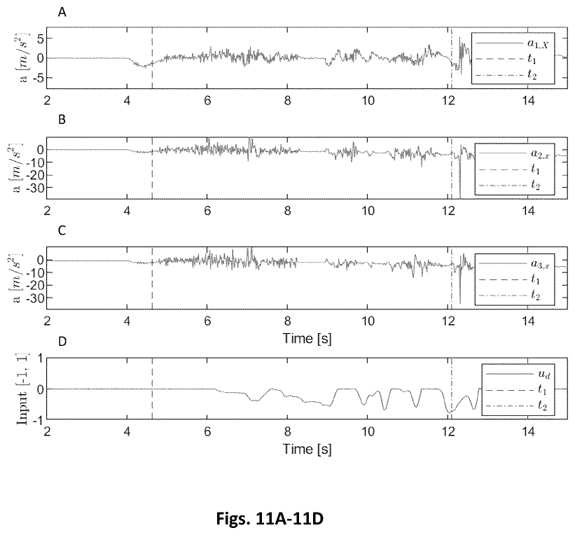

One component from each IMU was used for wavelet feature extraction and analysis. The three accelerations included a_{2,x} and a_{3,x} from IMU 2 and IMU 3, respectively, and the transformed acceleration component a_{1,x}=a_{1,x} cos α−a_{1,2} sin a from IMU 1 in the body frame of the loader (X, Z). (Note that the body coordinate frame (X, Z) was fixed to the front of the loader, so X is parallel to the ground.) The rotation angle α was estimated using

$$α = ( \frac{{\overline{a}}_{1 , x}}{{\overline{a}}_{1 , z}} ) , \qquad ( 18 )$$

where ā_{1,x} and ā_{1,z} are the mean acceleration values over the first 1 s of data. This transformation minimizes the impact of different boom IMU orientations due to errors in positioning the lift cylinder in the pile entry position (the first step in the excavation trial procedure). With this transformation, the acceleration in the X-direction in the body coordinate frame was used for IMU 1. Examples of acceleration and control inputs of a trial are shown in FIGS. 11A-11D respectively.

### Excavation Start Time

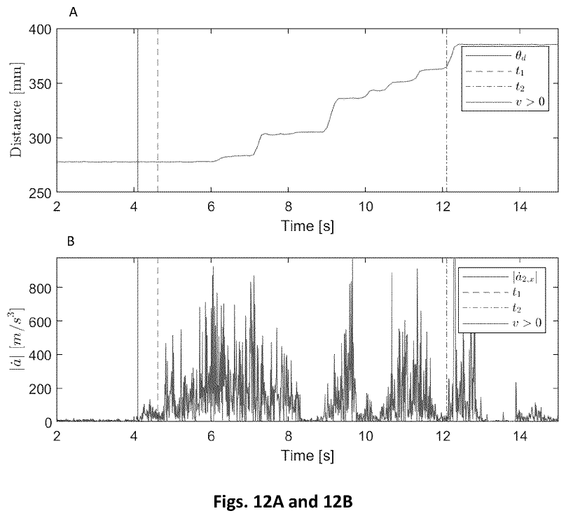

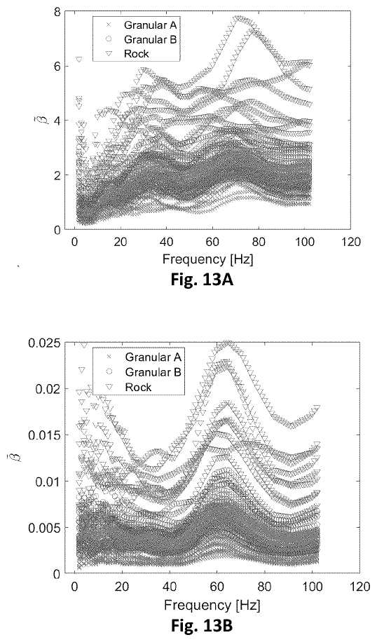

The first step was identifying the start time, τ₁, of the excavation cycle. The absolute value of the time derivative of the accelerometer signals, |\dot{a}|, was used to identify the time when a threshold, 2, had been reached. When the loader first moves forward toward the pile, the metric could potentially reach the threshold prematurely. To minimize the potential impact the search window starts 0.5 s after the loader started to move forward by inspection of the velocity estimate v. Through inspection of multiple trials, Q was found to be 45 m/s³. FIGS. 12A and 12B show examples of computing the excavation start time.

### Excavation Stop Time

The end of the excavation cycle, τ₂, was estimated using the dump cylinder extension, θ_{d}. The excavation cycle ends when the dump cylinder reaches the minimum of either 372 mm or when the dump cylinder reaches the extension value 15 s after τ₁. The first criteria ensures the induced vibration caused by the bucket reaching the mechanical stops is not contained within the excavation window. The second criteria overcomes the scenario when the operator fails to extend the dump cylinder fully during excavation. FIGS. 12A and 12B show examples of computing the excavation end time.

### Results

The material classification method was tested by first extracting wavelet features as described above from the segmented acceleration signals a_{1,x}, a_{2,x} and a_{3,x}. The features were then clustered using the k-means algorithm described above to test the classification performance for the three material classes (piles). MATLAB® 2020b (version 9.9) with Wavelet Toolbox (version 5.5) and Statistics and Machine Learning Toolbox (version 12.0) were utilized for this analysis. Size distribution estimates of the Rock and Granular B pile were generated using Granular A size distribution as the ground truth reference. The following subsections present the feature extraction, classification and size distribution estimatation results.

### Feature Extraction Using Wavelet Analysis

The normalized wavelet features, \tilde{β}(f), was resampled starting at 2 Hz, with 1 Hz increments, and stopping at 102 Hz. Thus, n=100 features were extracted from each acceleration signal. These features were stored as o₁₀₀ₓ₁ feature vectors to use as inputs for the classification algorithm. Note that the 1 Hz increment value is tunable and 1 Hz was selected as it captured the low frequency fluctuations while not oversampling the higher frequencies.

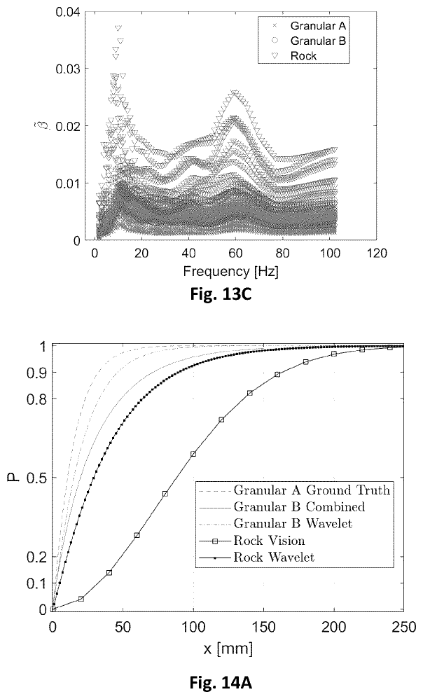

The features \tilde{β}(f) extracted from a_{1,x}, a_{2,x} and a_{3,x} for the 70 excavation trials are shown in FIGS. 13A-13C, respectively. An inspection of the wavelet features highlights Rock being distinguishable at higher frequencies relative to Granular A and Granular B; however Granular A and Granular B have some overlap. There does not exist one frequency that can distinguish all three piles.

Classification Using k-Means

The 70 feature vectors o were clustered using the k-means unsupervised learning algorithm. The algorithm was initialized with k=3 prototypes to form 3 clusters with the feature set when comparing Rock, Granular B, and Granular A and initialized with k=2 prototypes for binary classification. The classification performance using cluster purity (17) of different combinations of the three piles is given in Table 3 as the first number for each entry (the second number for each entry is the result of a preliminary classification based on fewer trials and features).

The feature sets from the three different acceleration signals were classified independently. In a practical implementation, the fewest number of sensors may be preferred to reduce costs and complexity, which is the motivation for each signal processed independently.

TABLE 3
Classification accuracy.

| Pile ID | a_{1, X}(%) | a_{2, x}(%) | a_{3, x}(%) |
|---|---|---|---|
| Rock & Granular B | 97, 92 | 95, 80 | 93, 80 |
| Rock & Granular A | 98, 93 | 98, 86 | 97, 92 |
| Granular B & Granular A | 82, 71 | 83, 86 | 87, 77 |
| Rock, Granular B, & Granular A | 80, 86 | 82, 83 | 80, 75 |

Inspection of Table 3 reveals the classification rates are similar between the three IMUs and the performance was high. Classification was highest for Rock and Granular A, which were the two piles with the largest difference in mean rock size.

### Size Distribution Estimation

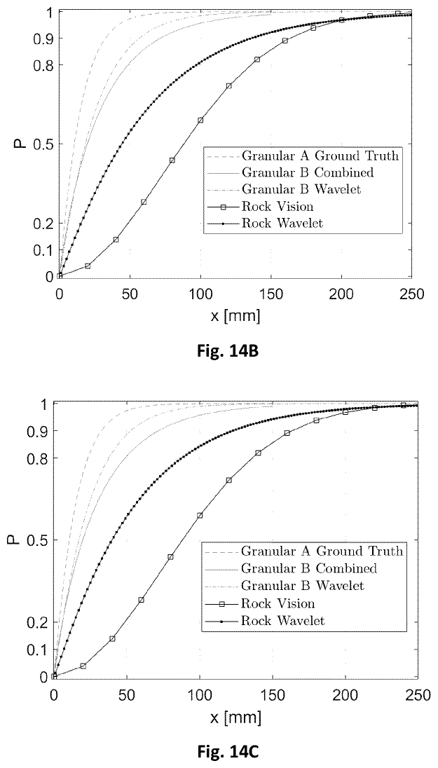

Granular A was used as the ground truth reference needed in (9). The uniformity parameter n for all three piles was assumed to be the same as the ground truth reference. Table 4 provides estimates of the mean rock size parameter for the two other piles using the wavelet features extracted from a_{1,X}, a_{2,x} and a_{3,x}. FIGS. 14A-14C show the estimated size distributions for Granular B and Rock using the wavelet features extracted from a_{1,x}, a_{2,x} and a_{3,x}, respectively.

TABLE 4
Mean size parameter estimates for Granular B and Rock.

| Parameter | a_{1, X} | a_{2, x} | a_{3, x} | Measured |
|---|---|---|---|---|
| x_{50, B} [mm] | 15.1 | 18.4 | 17.1 | 19.8 |
| x_{50, R} [mm] | 29.2 | 44.3 | 39.9 | 88 |

Inspection of Table 4 demonstrates the ability of the wavelet features to estimate the mean rock size for the Granular B pile. The measured value for Granular B was the mean size from the combined models of the screening and vision-based fragmentation analysis methods.

The mean size estimates for the Rock pile are approximately 50% less than the estimate value using a vision-based fragmentation analysis system. Because the ground truth reference was from a combination of size distribution models estimated from a screening and a vision-based fragmentation analysis system, discrepency is expected.

### Example 3. Acceleration Sensing and Wavelet Analysis

This example describes automatic aggregate material classification, using acceleration data obtained from manual loading of rock piles into a scale model haulage equipment, and wavelet analysis.

### Equipment

The model haulage equipment was a small-scale truck equipped with a steel bed with a volume of about 53 L. Two Microstrain® 3DM-GX5-25 AHRS inertial measurement units (IMUs) (LORD, MicroStrain® Sensing Systems, Williston, VT, USA) were mounted to the truck. One of the IMUs was mounted on the truck bed (tool) and the other IMU was mounted on the truck chassis. Material was dumped from a pail onto the truck bed. The sensors captured inertial signals during loading the truck bed.

The IMUs include a triaxial accelerometer, gyroscope, and magnetometer with a measurement ranges of ±8 g, ±300° /s, and ±8 Gauss respectively, with a sampling rate of up to 1000 Hz.

### Rock Piles

The material consisted of four rock piles (1-4) with different rock size distribution (pile 1 was the coarest material and pile 4 was the finest).

Piles 1-4 were assessed for size distribution. A qualitative assessment of the four rock piles showed that Piles 1 and 2 had similar fragmentation, Piles 3 and 4 contained smaller rocks than Piles 1 and 2, and Pile 4 contained the most fine sized rocks.

Processing was done using a size distribution model based on the Rosin-Rammler equation (8). To obtain a ground truth reference, material from the four piles was processed using a Gilson Testing Screen Model TS-1.

Material was placed on the top screen and the machine was set into operation for 5 minutes. The machine allows for six different sized sieve trays to be used. Sieve sizes were selected based on some a priori estimate of the fragmentation parameters to ensure individual trays did not become overloaded with material.

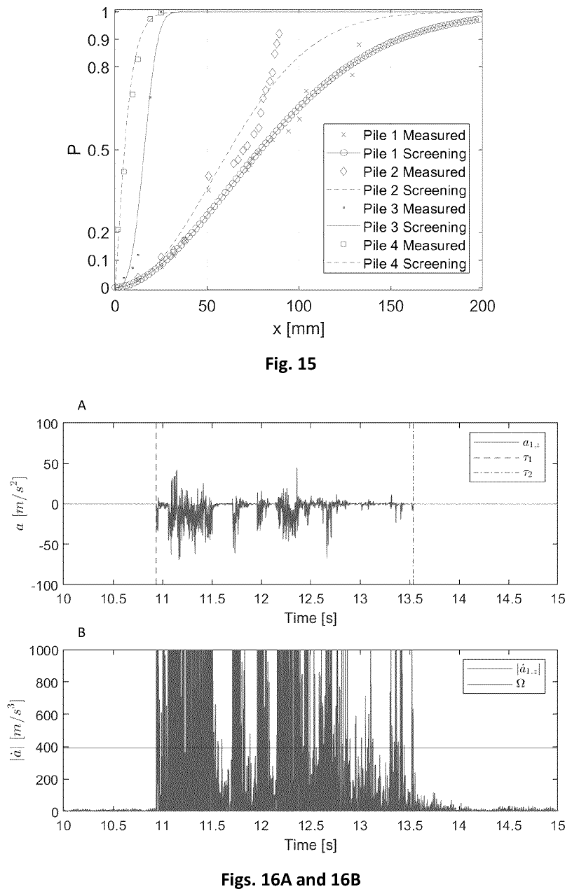

The largest sized sieve tray available was 50 mm and Piles 1 and 2 contained a significant portion of rocks much larger than 50 mm. Oversized rocks, those that were visually larger than 5 cm, were removed and weighed individually. Estimating the size of the oversized rocks was accomplished using the Ontario Provincial Standard Specification (OPSS) numbered 1004 (OPSS.PROV 1004, Aggregates-Miscellaneous) as a reference. Results from the Gilson TS-1 were combined with the manually measured oversized rocks to create a data set for the size distribution parameters x₅₀ and n for each of the four piles, shown in Table 5 as the first number for each entry (the second number for each entry is the result if the oversized particles were grouped using the rock size grouping referenced in OPSS.PROV 1004, which would reduce the number of data points for oversize rocks). FIG. 15 is a plot of the fragmentation measurements and the corresponding Rosin-Rammler size distribution model estimate for the four piles. Inspection of FIG. 15 reveals the fragmentation measurements for Pile 2 indicate a mean rock size of 70 mm whereas the model estimate has a value of 62.2 mm, a 11% discrepency size distribution model for Pile 2

TABLE 5
Estimated Rosin-Rammler size distribution model
parameters x₅₀ and n for Piles 1-4 using screens.

| Rock Pile | x₅₀ [mm] | n |
|---|---|---|
| 1 | 79.0, 66 | 1.7989, 2,1259 |
| 2 | 62.2, 55 | 2.0319, 2.5517 |
| 3 | 15.8, 17 | 3.1215, 2.9518 |
| 4 | 5.0, 5 | 1.2209, 1.2712 |

### Analyses

Equations (1)-(5) were used for the wavelet analysis and the continuous wavelet transform function in MATLAB®. For the analysis a k-means unsupervised learning algorithm (16) was used for the different material classes. Size distribution estimates was generated using equation (9).

A total of 20 trials with Pile 1, 18 trials with Pile 2, 7 trials with Pile 3 and 19 trials with the Pile 4 were performed.

### Acceleration Signals

One component from each IMU was used for wavelet feature extraction and analysis. The two accelerations included a_{1,z} and a_{2,z} -from IMU 1 (mouted on the haul bed) and IMU 2 (mounted on the chassis), respectively. Each acceleration signal is represented in discrete time as f[k]=f(kT), where k=0,1,2, . . . L is the time-step and T is the sampling time (0.001 s). The signal length recorded for each trial was consistent with L=30000, representing 30 seconds of data which ensured the loading signal was captured within the 30 second time window.

### Loading Start Time

The first step was identifying the start time, τ₁, of the loading cycle. The absolute value of the time derivative of the accelerometer signals, |\dot{a}|, was used to identify the instance when a threshold, Ω, has been reached. Through inspection of multiple loading trials Ω was found to be 196.2 m/s³. FIGS. 16A and 16B provide an example of computing the loading start time.

### Loading End Time

The end of the loading cycle, τ₂, was estimated using |\dot{a}| and the threshold Ω by assessing the signal from the end (k=30000) and working backwards to k=1 until the threshold was reached. Through inspection of multiple loading trials Q was found to be 392.4 m/s³ for Piles 1 and 2 and 196.2 m/s³ for Piles 3 and 4. FIGS. 16A and 16B show examples of computing the loading end time.

Feature Extraction using Wavelet Analysis

The segmented acceleration signal was then processed using equations (1)-(5) described in the embodiments. The normalized wavelet result, \tilde{β}(f), was resampled starting at 15 Hz, with 10 Hz increments, and stopping at 405 Hz. Thus, n=40 features were extracted from each acceleration signal. These features were stored as o₄₀ₓ₁ feature vectors to use as inputs for the classification algorithm. Note that the 10 Hz increment value is tunable and 10 Hz was selected as it captured the low frequency fluctuations while not oversampling the higher frequencies.

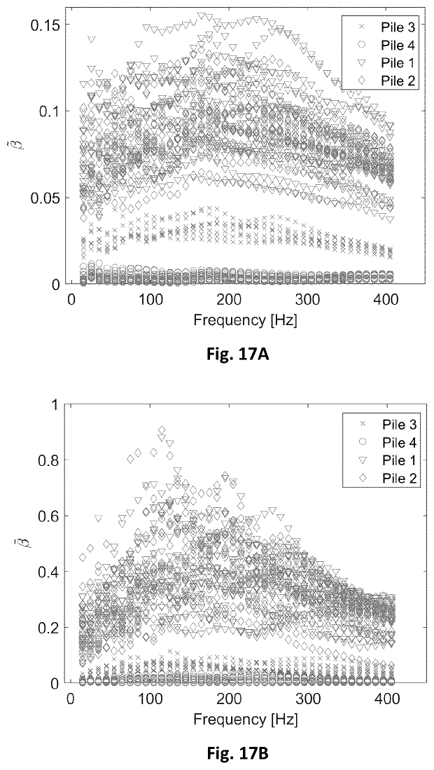

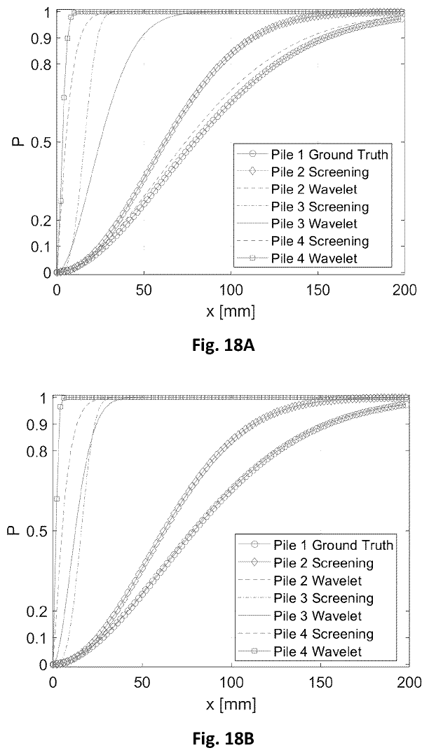

The features \tilde{β}(f) extracted from a_{1,z} and a_{2,z} for the 64 loading trials are shown in FIGS. 17A and 17B, respectively. At higher frequencies the features appear to distinguish Pile 3 and 4 from Piles 1 and 2 where Piles 1 and 2 appear to be similar. There does not exist one frequency that can distinguish all four rock piles.

Classification Using k-Means

The 64 feature vectors o were clustered using the k-means unsupervised learning algorithm (16). The algorithm was initialized with k=2,3,4 prototypes to form clusters when comparing different pairing of rock piles shown in Table 6. The second number for the last entry is the result of a preliminary classification based on fewer trials.

TABLE 6
Classification accuracy.

| Rock Piles | a_{1, z}(%) | a_{2, z}(%) |
|---|---|---|
| 1 & 2 | 62 | 54 |
| 1 & 3 | 83 | 90 |
| 1 & 4 | 100 | 100 |
| 2 & 3 | 96 | 89 |
| 2 & 4 | 100 | 97 |
| 3 & 4 | 100 | 100 |
| 1, 2 & 3 | 66 | 56 |
| 1, 2 & 4 | 70 | 69 |
| 1, 3 & 4 | 85 | 73 |
| 2, 3 & 4 | 89 | 84 |
| 1, 2, 3 & 4 | 70, 77.8 | 65, 70.4 |

Inspection of Table 6 shows the classification rate of the haul bed mounted IMU performing slightly better than the chassis mounted IMU. Classification of Piles 1 and 2 yielded the lowest classification accuracy.

### Size Distribution Estimation

Pile 1 was used as the ground truth reference needed in (9). The uniformity parameter for all four piles is assumed to be the same as the ground truth reference. Table 7 provides estimates of the mean rock size parameter for the three other piles using the wavelet features extracted from a_{1,z}, and a_{2,z}. FIGS. 18A and 18B shows the estimated size distributions using wavelet features.

TABLE 7
Mean size parameter estimates for Piles 2-4.

| Rock Pile | Parameter | a_{1, z} | a_{2, z} | Measured |
|---|---|---|---|---|
| 2 | x₅₀ [mm] | 73.9 | 77.9 | 62.2 |
| 3 | x₅₀ [mm] | 24.8 | 12.5 | 15.8 |
| 4 | x₅₀ [mm] | 3.1 | 1.7 | 5.0 |

Inspection of Table 7 demonstrates the ability of the wavelet features to estimate the mean size x₅₀ with good results. Recalling from the inspection of FIG. 15 the estimated size distribution model for Pile 2 underestimated the mean rock size. Deviation from the measured value could be due to manually dumping the piles where the dumping height may have fluctuated as well as the dumping speed of the bucket, could be from a poor screening analysis, or that the rock size changed after each trails due to breakage. However, the results demonstrate that with the many potential sources of variability the wavelet features were still successful classifying the piles using k-means and the size distribution mean size parameter estimates yielded good results.

### Example 4. Force Sensing and Wavelet Analyis

This example demonstrates the scalability of the invention through rock size distribution estimation using force data and wavelet features extracted from the force data. The force signal was acquired during manual excavations using the Kubota R520s 1-tonne capacity wheel loader as described in Example 2 and using the Epiroc ST14 14-tonne capacity load haul dump machine as described in Example 1. The wavelet features combined with a ground truth reference are able to accurately estimate the mean size of each of the rock piles.

The Kubota R520s wheel loader was outfitted with pressure sensors (Measurement

Specialties, measurement range 0 to 350 Bar, analog voltage range 0.5 to 4.5 VDC, accuracy<±0.05% F.S) placed at the head (h) and rod (r) sides of the machine's lift cylinders similar to the ST14 pressure sensors shown in FIG. 2. These pressure sensors were used to measure the cylinder hydraulic pressures P at the cylinder head side (h) and rode side (r). Using the cylinder head side and rod side cross-sectional areas A, the interaction forces were estimated using f=PₕAₕ−PᵣAᵣ. The pressure signal was sampled at 250 Hz.

### Rock Piles

Five rock piles were used to collect the force signal during excavation. Three piles (named Granular A, Granular B and Rock) were used with the 1-tonne capacity wheel loader and two piles (named Muck and Gravel) were used with the 14-tonne capacity load haul dump machine.
Rock: described in Example 2 as Rock.
Granular B: described in Example 2 as Granular B.
Granular A: described in Example 2 as Granular A.
Muck: described in Example 1 as rock with size distribution model parameters of x₅₀=362.5 mm and n=2.1474. The model parameter estimates were produced using WipFrag®, a vision-based fragmentation analysis system, and an image of the pile.
Gravel: described in Example 1 as gravel with size distribution model parameters of x₅₀=141.3 mm and n=1.7938. The model parameter estimates were produced using WipFrag® and an image of the pile.

### Analyses

Equations (1)-(5) were used for the wavelet analysis and the continuous wavelet transform function in MATLAB®. Size distribution estimates was generated using equation (9).

A total of 30 mannual excavation trials with the Granular A pile and 30 manual exavation trials with the Granular B pile were performed using the 1-tonne capacity Kubota R520s wheel loader.

Muck and Gravel autonomous excavation experiments discussed in Example 1 using the Epiroc ST14 LHD were filtered to trials where uₜ>0 during the digging phase. This resulted in 25 trials for the Muck pile and 25 trials for the Gravel pile. The wavelet features require the velocity of the machine during digging to be approximately the same for each excavation trial. When uₜ=0 the velocity reduced significantly and thus the trial was not included.

### Signal Processing

The force signals were filtered using a high-pass filter with a passband frequency of f_{cut}=0.5 Hz. The MATLAB function highpass was used to perform the high-pass filtering on the force signals. A highpass filter was used to extract only the forces due to the momentum change or impulse as described in the embodiments. The force signal contains both a static force due to gravity as well as the force due to the impulse, using the highpass filter effectively removed the contribution due to gravity.

### Segmentation

The filtered force signals were segmentation to f[τ₁, τ₂], where τ₁ and τ₂ are the start and stop times described in Example 2 for the Kubota R520s data and to f[0, τ₂] for the ST14 data as described in Example 1.

### Normalization

An estimate of the payload (mass of material in the bucket) for each excavation trial was used to normalize (5).

### Results

Size distribution estimates were generated using the wavelet features described above from the filtered and segmented force signals. The ratio of mean wavelet features along with a ground truth reference were able to accurately predict the size distribution of rock piles. The following subsections present the feature extraction and size distribution estimate results.

### Feature Extraction Using Wavelet Analysis

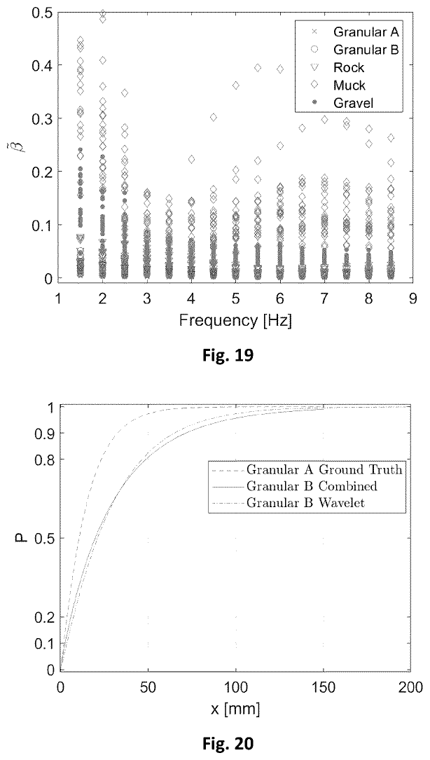

The normalized wavelet result, \tilde{β}(f), was resampled starting at 1.5 Hz, with 0.5 Hz increments, and stopping at 8.5 Hz. Thus, n=15 features were extracted from the force signal and are shown in FIG. 19.

### Size Distribution Estimation

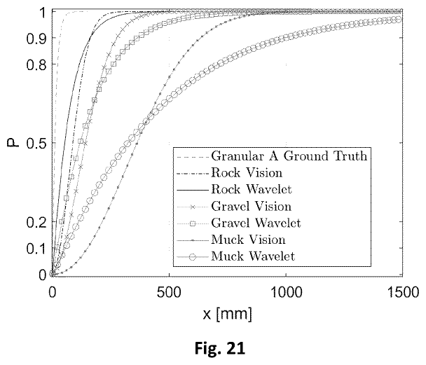

Granular A was used as the ground truth reference needed in (9). The uniformity parameter for all four piles is assumed to be the same as the ground truth reference. Table 8 provides estimates of the mean rock size parameter for the four other piles using the wavelet features extracted from f. FIG. 20 shows the estimated size distribution for Granular B using the wavelet features and the combination of a screening and vision-based fragmentation analysis systems. FIG. 21 shows the estimated size distributions for Rock, Muck and Gravel using the wavelet features and a vision-based fragmentation analysis system.

TABLE 8
Mean size parameter estimates for
Granular B, Rock, Muck and Gravel.

| Pile Name | Parameter | f | Measured |
|---|---|---|---|
| Granular B | x₅₀ [mm] | 21.1 | 19.8 |
| Rock | x₅₀ [mm] | 52.7 | 88 |
| Muck | x₅₀ [mm] | 324.4 | 362.5 |
| Gravel | x₅₀ [mm] | 118.6 | 141.3 |

Inspection of Table 8 identifies the ability of the wavelet features to estimate the mean size X₅₀ with good results especially since the wavelet features used were obtained from two different machines.

The mean size estimates for the Rock, Muck and Gravel underestimate the estimated mean size using a vision-based fragmentation analysis system. Because the ground truth reference was from a combination of size distribution models estimated from a screening and a vision-based fragmentation analysis system, discrepency is excpected. A common drawback to vision-based fragmentation analysis systems are the poor identification of small rocks leading to a higher size estimate.

### Equivalents

While the invention has been described with respect to illustrative embodiments thereof, it will be understood that various changes may be made to the embodiments without departing from the scope of the invention. Accordingly, the described embodiments are to be considered merely exemplary and the invention is not to be limited thereby.

1. A method for classifying excavation media, comprising:
obtaining sensor signals from one or more proprioceptive sensors on a machine interacting with the material;
using a processor to process the sensor signals, wherein processing includes extracting features from the sensor signals;
selecting one or more classification categories corresponding to physical characteristics of the material;
using the extracted features as inputs to a classifier;
wherein the classifier uses one or more algorithms to classify the extracted features into the selected classification categories; and
outputting a result indicating at least one classification category relating to a physical characteristic of the material.

2. The method of claim 1, wherein the excavation media comprises fragmented rock, gravel, sand, soil, or mixtures thereof.

3. The method of claim 1, wherein the one or more proprioceptive sensor comprises at least one of a force sensor, a pressure sensor, an inertial measurement unit (IMU) sensor, a displacement (linear, angular) sensor, a current sensor and a voltage sensor.

4. The method of claim 1, wherein the machine comprises an excavator, haulage equipment, a load haul dump (LHD) machine, or a conveyor.

5. The method of claim 1, wherein the machine is operating in an application selected from underground mining, surface mining, construction, material handling, material preparation, and space exploration and development.

6. The method of claim 1, wherein the machine interacts with the material manually, partially autonomously, or fully autonomously.

7. The method of claim 1, wherein processing the sensor signals and extracting features includes an analysis with respect to time, frequency, amplitude, or a combination thereof.

8. The method of claim 1, wherein processing the sensor signals and extracting features includes a statistical analysis, a stochastic analysis, a fractal analysis, a wavelet analysis, a spectral analysis, or a combination of two or more thereof.

9. The method of claim 1, wherein the classifier uses supervised learning to classify the identified features according to the selected classification categories.

10. The method of claim 1, wherein the classifier uses unsupervised learning to classify the identified features according to the selected classification categories.

11. The method of claim 1, wherein the classifier uses unsupervised and supervised learning to classify the identified features according to the selected classification categories.

12. The method of claim 1, further comprising obtaining and processing sensor signals from one or more exteroceptive sensors.

13. The method of claim 12, wherein the one or more exteroceptive sensors are selected from cameras and laser scanners.

14. Apparatus for classifying material, comprising:
an input device that receives at least one sensor signal from at least one proprioceptive sensor of a machine interacting with the material;
a processor that:
processes the at least one sensor signal and extracts features in the at least one sensor signal;
selects one or more classification categories corresponding to physical characteristics of the material
identifies extracted features of the at least one sensor signal that are similar for each selected classification category of the material;
uses the identified features as inputs to a classifier that uses an algorithm to classify the identified features into the selected classification categories; and
outputs a result indicating at least one classification category relating to a physical characteristic of the material.

15. The apparatus of claim 14, wherein the excavation media comprises fragmented rock, gravel, sand, soil, or mixtures thereof.

16. The apparatus of claim 14, wherein the at least one proprioceptive sensor comprises a force sensor, a pressure sensor, an inertial measurement unit (IMU), a displacement (linear, angular) sensor, a current sensor, or a voltage sensor.

17. The apparatus of claim 14, wherein the machine comprises an excavator, wheel loader, haulage equipment, a load haul dump (LHD) machine, or a conveyor.

18. The apparatus of claim 14, wherein the machine is operating in an application selected from underground mining, surface mining, construction, material handling, material preparation, and space exploration and development.

19. The apparatus of claim 14, wherein the machine interacts with the material manually, partially autonomously, or fully autonomous.

20. The apparatus of claim 14, wherein processing the sensor signals and extracting features includes an analysis with respect to time, frequency, amplitude, or a combination thereof.

21. The apparatus of claim 14, wherein processing the sensor signals and extracting features includes a statistical analysis, a stochastic analysis, a fractal analysis, a wavelet analysis, a spectral analysis, or a combination of two or more thereof.

22. The apparatus of claim 14, wherein the classifier uses supervised learning to classify the identified features according to the selected classification categories.

23. The apparatus of claim 14, wherein the classifier uses unsupervised learning to classify the identified features according to the selected classification categories.

24. The apparatus of claim 14, wherein the classifier uses unsupervised and supervised learning to classify the identified features according to the selected classification categories.

25. The apparatus of claim 14, further comprising one or more exteroceptive sensors.

26. The apparatus of claim 25, wherein the one or more exteroceptive sensors are selected from cameras and laser scanners.

27. Non-transitory computer readable storage media compatible with a computer, the storage media containing instructions that, when read by the computer, direct the computer to carry out processing steps comprising one or more of:
processing sensor signals from one or more proprioceptive sensors disposed on a machine interacting with a material;
wherein processing includes extracting features from the sensor signals;
selecting one or more classification categories corresponding to physical characteristics of the material;
using the extracted features as inputs to a classifier;
wherein the classifier uses one or more algorithms to classify the extracted features into the selected classification categories; and
outputting a result indicating at least one classification category relating to a physical characteristic of the material.

28. The non-transitory computer readable storage media of claim 27, wherein the classifier uses one or more algorithms selected from a supervised learning algorithm and an unsupervised learning algorithm, or a combination thereof.

29. The non-transitory computer readable storage media of claim 27, wherein the classifier uses one or more supervised learning algorithm selected from a k-nearest neighbour (KNN) algorithm and an artificial neural network (ANN) algorithm, or a combination thereof.
